(19) United States
(12) Patent Application Publication
(10) Pub. No.: US 2004/0171935 A1
(43) Pub. Date: Sep. 2, 2004

(54) Ultrasound transducer probe identification for security and other purposes

(75) Inventors: Donald L. Van Creveld, Menlo Park, CA (US); Timothy H. Hauser, North Bend, WA (US); Mirsaid Bolorforosh, Portola Valley, CA (US); Sean C. Little, San Jose, CA (US); Vaughn R. Marian, Saratoga, CA (US); Lars Becker, Los Altos, CA (US)

Correspondence Address: Siemens Corporation, Intellectual Property Department, 170 Wood Avenue South, Iselin, NJ, 08830, US

(73) Assignee: Siemens Medical Solutions USA, Inc.

(21) Appl. No.: 10/799,453

(22) Filed: Mar. 12, 2004

Related U.S. Application Data
(63) Continuation-in-part of application No. 10/185,217, filed on Jun. 27, 2002.

(51) Int. Cl. A61B 5/05

(52) U.S. Cl. 600/437

(57) ABSTRACT

Transducer probe identification is provided. A wireless identification is provided. For example, a radiofrequency identification tag is positioned within a transducer probe. A receiver is positioned within the imaging device. Using the wireless information, the imaging device may generate a list of available transducers regardless of whether they are connected with the imaging device. As an alternative or addition, information specific to a transducer is provided to the imaging device. Using either wireless transmission or a wired connection, the information distinguishes a given transducer from other transducers of a same type and manufacturer or from all other transducers. The specific identifier is then used with security information to prevent theft. For example, a code from the transducer probe is compared with a security code in a list on the imaging device, entered in response to a request generated once the transducer probe is connected to the imaging device, communicated from an adapter that connects with the transducer probe and the imaging device or other sources of security information. If the security information does not match the transducer code, the imaging device, the transducer probe or both may be disabled. Alternatively, a warning is displayed, such as “This transducer probe is the property of Hospital.”

### REFERENCE TO RELATED APPLICATIONS

The present patent document is a continuation-in-part of application Ser. No. 10/185,217, filed Jun. 27, 2002, which is hereby incorporated by reference.

### BACKGROUND

The present invention relates to ultrasound transducer probe identification. In particular, transducer probe identification is used for security or may be communicated to an imaging system more desirably.

In medical diagnostic ultrasound, transducer probes are releasably connectable with imaging devices. The releasable connection allows different types of transducer probes to be connected to the same imaging device. The transmit and receive beamforming as well as other processes implemented within the imaging device are performed differently for different types of transducer probes. For automatic assistance in configuring the imaging device, the transducer probes are hard wired with a code or other electrical indication of the type of transducer probe. For example, a cardiology probe having a certain number of transducer elements has a series of signal lines, some short circuited and others open circuited. The series of signal lines provides a code indicating the type of transducer. U.S. Pat. No. 6,270,460 shows one such system using fuses to allow further coding based on the number of uses of a catheter based transducer probe. However, the imaging device only senses the type of transducer probe once the transducer probe is connected to the imaging device.

Transducer probes are expensive, costing up to tens of thousands of dollars. The transducer probes are operable with different ultrasound imaging devices, such as a same type of imaging device made by a same manufacturer. To avoid the cost associated with purchasing a transducer probe, some individuals may acquire transducer probes in an inappropriate manner. Transducer probes may be accidentally removed from one facility and taken to another facility by sonographers or doctors performing ultrasound examinations at both locations. Whether intentional or accidental, the loss of transducer probes is expensive. While serial number information provided on a sticker or engraved or molded into a transducer probe may allow for tracking of the transducer probe, such information may easily be altered, removed or ignored. Other than locking transducer probes within a box, cabinet or room, little assistance is currently provided in preventing loss of transducer probes.

### BRIEF SUMMARY

By way of introduction, the preferred embodiments described below include methods and systems for transducer probe identification. Rather than a hard wired identification of a transducer probe, a wireless identification is provided. For example, a radiofrequency identification tag (RFID) is positioned within a transducer probe. A transceiver is positioned within the imaging device to transmit an interrogation request to the remotely located transducer probe. The RFID responds to the request with a coded radio frequency transmission that identifies the type or specific transducer probe. Using the wireless information from a multitude of transducer probes, the imaging device may generate a list of available transducers regardless of whether they are connected with the imaging device. Programmable data in addition to identification information may be wirelessly provided for populating exam information. As an alternative or addition, information specific to a transducer is provided to the imaging device. Using either wireless transmission or a wired connection, the information distinguishes a given transducer from other transducers of a same type and manufacturer or from all other transducers, depending on the size of the identification code used. The specific identifier is then used with security information to prevent theft. For example, a code from the transducer probe is compared with a security code provided in a list archived in the imaging device, entered in response to a request generated once the transducer probe is connected to the imaging device, communicated from an adapter that connects with the transducer probe and the imaging device or other sources of security information. If the security information does not match the transducer code, the imaging device, the transducer probe or both may be disabled. Alternatively, a warning is displayed, such as “This transducer probe is the property of Hospital” or “This transducer is not registered for operation with the current imaging device” or other such warnings.

Various aspects and advantages of the preferred embodiments discussed below or summarized above may be now or later claimed independently or in combination. In a first aspect, a system is provided for identifying unauthorized use of a transducer which is detachably connectable with an imaging device. An electronic identifier is provided with the transducer. A processor is operable to generate a security signal in response to the electronic identifier.

In a second aspect, a method is provided for identifying unauthorized use of a transducer which is detachably connectable with an imaging device. A transducer code is compared with a security code. A security signal is generated in response to the comparison. For example, a security signal indicates authorized use. As another example, the security signal indicates unauthorized use.

In a third aspect, a system for transducer identification is provided. The system includes a transducer. A wireless identifier tag connects with the transducer.

In a fourth aspect, an improvement in a method for transducer identification is provided. The transducer is electronically identified by an imaging device. The improvement is transmitting identification information wirelessly from the transducer.

In a fifth aspect, an improvement in a method for electronically identifying transducer information once connected to an imaging device is provided. The transducer is electronically distinguished from other transducers of a same type and manufacturer.

The present invention is defined by the following claims and any later added claims, and nothing in this section should be taken as a limitation on those claims. Further aspects and advantages are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for transducer identification;

FIG. 2 is a partial block diagram of another embodiment of a transducer identification system;

FIG. 3 is a flow chart of one embodiment of a method for providing transducer probe security information; and

FIG. 4 is a flow chart diagram of one embodiment of a method for providing identification information wirelessly.

### DETAILED DESCRIPTION OF THE DRAWINGS AND PRESENTLY PREFERRED EMBODIMENTS

Two features included herein are wireless transmission of identification information for a transducer probe and relatively unique transducer probe identification to allow security schemes for preventing theft. Both features may be provided in a same embodiment, but may also be provided in separate embodiments.

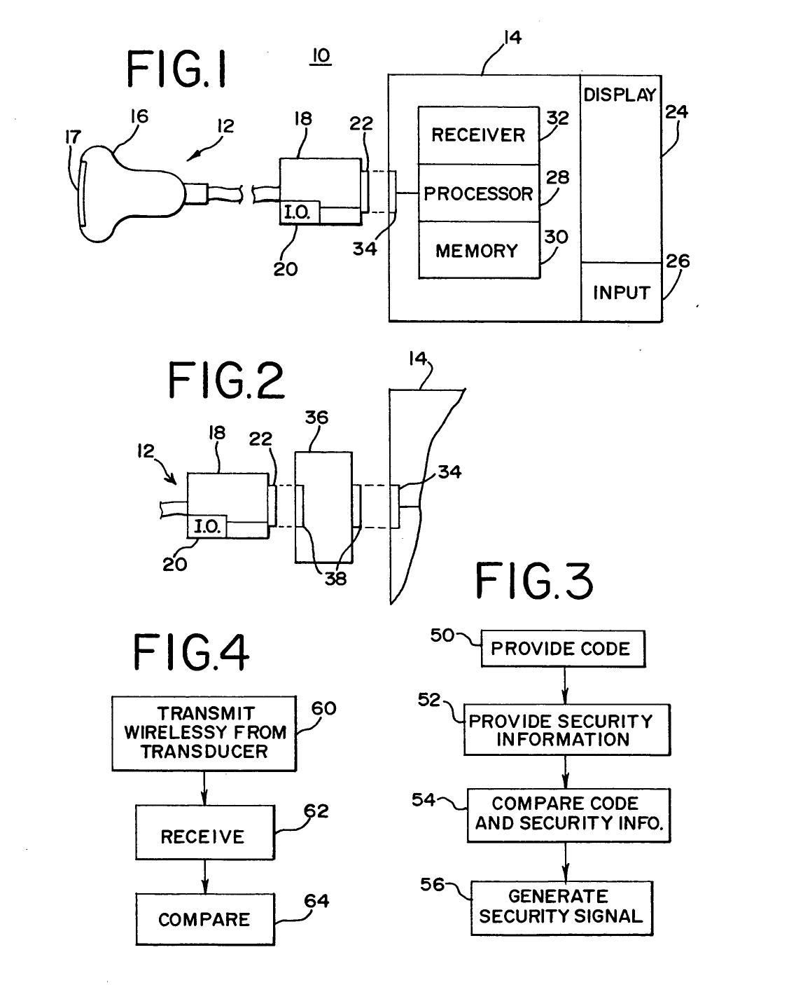

FIG. 1 shows a system 10 for transducer identification. The system 10 includes a transducer assembly 12 and an imaging device 14. Additional, different or fewer components may be provided. For example, a plurality of different transducer assemblies 12 is provided for use with a same or a group of imaging devices 14.

The transducer probe assembly 12 is one or more of any now known or later developed transducer probe assemblies. As shown, a hand-held transducer probe assembly for releasable connection with an imaging device 14 is provided. In other embodiments, an endocavity, catheter or other transducer probe is used. In yet another embodiment, the transducer probe assembly 12 without the cable is provided for releasable connection with the imaging system 14, such as for transducer probe assemblies connectable with hand-held imaging systems. The embodiment of the transducer probe assembly 12 shown in FIG. 1 includes a transducer probe 16, a connector housing 18, an electronic identifier 20 and a connector 22. Additional, different or fewer components may be provided, such as including additional electronics within the transducer probe 16 or the connector housing 18.

The transducer probe 16 includes a transducer 17. The transducer 17 is a one-dimensional or multidimensional array of piezoelectric or capacitive membrane transducer elements. For medical diagnostic ultrasound imaging, the transducer 17 generates acoustic energy which is coupled into the body. The same transducer 17 receives acoustic energy from the internal organs and converts the energy to electrical signals for interpretation by the imaging device. Electrical signals are provided to and from the transducer 17 to the imaging device 14 during use. For use, the transducer assembly 12 is connected with the imaging device 14.

The connector housing 18 includes the electronic identifier 20 and the connector 22. The connector housing 18 is a metal, plastic, rubber, polymer or other now known or later developed material for housing electrical and physical structure of the connector 22.

The connector 22 releasably connects using latches, grooves, tongues, or other now known or later developed physical and electrical releasable connections. The connector 22 includes a plurality of electrical connections to connect with a processor 28 of the imaging device 14. Some of the electrical connections provide electrical signals to and from the transducer 17 to the imaging device 14. In one embodiment, one or more electrical connections also connect the electronic identifier 20 with the imaging device 14. In alternative embodiments, the electronic identifier 20 transmits information wirelessly to the imaging device 14.

The electronic identifier 20 is a plurality of fuses selectively shorted or remaining intact to generate identification information, hardwired shorts and open connections, switches, nonvolatile memory, EEPROM, ROM, RAM, NVRAM, a memory, a processor, an application specific integrated circuit, transistors, digital device, analog device or other now known or later developed electronic device for outputting identification information, such as a digital code or analog signal having detectable characteristics. In the embodiment shown in FIG. 1, the electronic identifier 20 is within the connector housing 18, but may be positioned elsewhere within the transducer assembly 12 (e.g., in the transducer probe 16) or on an exterior portion of the transducer assembly 12. The electronic identifier 20 includes a code in either analog or digital form as an identifier to distinguish the transducer 17, transducer probe 16 or transducer probe assembly 12 from other transducers of a same type and manufacturer. For example, an identifier that repeats every 16,000, 32,000, 1 million or other number of times is provided during manufacture. The code or identifier is relatively unique to the particular transducer, rather than just being unique to a type of transducer and manufacturer. While repeat of the code may be provided, the distribution of transducer probes is such that repeat code for use on a same imaging device 14 is unlikely. Alternatively, each electronic identifier has an identification that is absolutely unique through the assignment of the code. In an alternative embodiment, the electronic identifier 20 identifies the type of transducer, but is otherwise not unique to the specific transducer assembly 12. Other levels of identification may be provided.

In an alternative embodiment, the electronic identifier 20 is a transmitter connected with a memory storing the code. For example, a radiofrequency or wireless identifier tag is used. Wireless identifier tags include a transponder having a code identifying the transducer, type of transducer or other information. The transponder includes a transmitter or integrates a global positioning system (GPS), local positioning system (GPS like), global system of mobile communication (GSM) or similar positioning systems. In response to an electric, magnetic or other field generated by the imaging device 14 or elsewhere, the transponder responds by wirelessly transmitting the code. In the transponder embodiment, an antenna emits radio signals to activate the wireless tag. The emitted radio signals may elicit a response only or may include information to be transmitted to a wireless tag. In one embodiment, the electromagnetic field generated by the antenna by a transceiver within the imaging device 14 is constantly generated, but may be activated in response to sensing of a proximate electronic identifier 20 or other sensor. Any of various frequency ranges may be used for the electromagnetic field, such as 30 to 500 Kilohertz, 150 to 950 MHz, 2.4 to 2.5 Gigahertz or other frequency ranges. The transceiver of the imaging device 14 may generate radio waves adapted in amplitude or other characteristic for operation within a limited area, such as within a typical room size for sonography. When the electronic identifier 20 passes within the electromagnetic zone, the electromagnetic activation is detected. In response, the electronic identifier 20 transmits the code. The code is then decoded by a receiver 32 within the imaging device 14. In other embodiments, the electronic identifier 20 transmits the code without using a transceiver, transponder or without responding to received information.

The radio frequency identification tag is either an active or a passive device. An active tag is powered by an internal battery within the transducer assembly or by external power provided through the connector once connected to the imaging device. The use of an active tag may allow for reading, writing or modification of data for the code stored and transmitted by the tag and sending data. For example, information regarding a patient being scanned where the scan application is transmitted to the electronic identifier 20. The electronic identifier 20 then stores the information in a memory for later transmission to a different imaging device 14. As another example, data from other sources within the transducer probe, such as a shock sensor, are stored for later transmission.

Passive radio frequency identification tags receive electromagnetic energy transmitted by the imaging device 14. They store and use this energy to power the transmission of the unique code which is intercepted by the imaging device 14 or some other device. The passive tag is a read-only device for outputting identification data or other data without modification in one embodiment, but a passive readable or writable tag may be used.

Additional information or coding may be transmitted wirelessly or through the connector. For example, micro-code or other information disclosed in U.S. Pat. No.______ (Ser. No. 10/185,217) is provided to the imaging device 14, such as to provide software or code to operate the imaging device 14 with the type or particular transducer assembly 12. As another example, image data is stored within the transducer assembly 12 and later transmitted.

The imaging device 14 is a medical diagnostic ultrasound scanner in one embodiment. Using signals or other information from the transducer assembly 12, the imaging device 14 generates a one-dimensional, two-dimensional or three-dimensional representation. Any now known or later developed imaging devices may be used, such as cart mounted, hand held, mobile, stationary or portable imaging devices. The imaging device 14 includes a display 24, an optional input 26, a processor 28, an optional memory 30, an optional receiver 32 and a connector 34. Additional, different or fewer components may be provided.

The receiver 32 is a transceiver in one embodiment. The receiver 32 generates an electromagnetic field for operation with a radiofrequency tag. Alternatively, a transceiver may transmit an inquiry or solicit a response from an identification tag 20 without generating a constant electric field. The receiver 32 may be a receiver without transmit capabilities in other embodiments. The receiver 32 is operable to receive the code from a transmitter, such as the electrical identifier 20. In alternative embodiments, a hardwire connection through the connectors 22 and 34 is provided for receiving a code from the electronic identifier 20, and the wireless receiver 32 is not provided. While shown within the imaging device 14, the receiver 32 may be positioned outside of the imaging device 14, such as on the imaging device 14 or remote from the imaging device 14. Using wireless or wired connection, the receiver 14 provides information to the imaging device 14. Alternatively, the receiver 32 interacts with other processors or devices without interacting with the imaging device 14. For example, the receiver 32 is operable to cause the transducer assembly 12 to be enabled or disabled using electrical or mechanical components within the transducer assembly 12.

The display 24 is an LCD, CRT, plasma, projector, monitor or other now known or later developed display. The display 24 outputs ultrasound images generated with signals from the transducer 17. Additional user interface features may be output on the display 24, such as request for security information, security information lists, lists of transducer types, specific transducers available, information about a transducer connected to the imaging system 24, the identification or other information provided by the electronic identifier 20, or other information.

The input 26 is a keyboard, mouse, track ball, touchpad, or other now known or later developed device for user entry of information through manipulation. Alternatively, the input 26 is a magnetic or optical disk drive for the input of machine readable data. The input 26 is operable to receive security information, such as an authorization or access code, a list of access or authorization codes, or other user information.

The processor 28 is an application specific integrated circuit, a general processor, a control processor, a digital signal processor, an encoder, a plurality of processors, an analog circuit, a digital circuit, combinations thereof or any other now known or later developed devices for identifying, using, selecting or otherwise operating on identification information from the electronic identifier 20. Software implemented by the processor 28 performs any one or more of the various security or other functions discussed herein. The processor 28 is connected through the connector 34 or wirelessly connected through the receiver 32 to the electronic identifier 20. In one embodiment, the processor 28 decodes the code from the electronic identifier 20. The information is decoded in response to a query or by reading information output by the electronic identifier 20. The processor 28 may configure the imaging device as a function of the type of transducer identified. Other processes using information provided from the transducer electronic identifier 20, such as populating fields of diagnosis reports, generating a relevant display identifying a transducer type, identifying an owner name or other information associated with the transducer assembly 12 or other configuration information may be performed by the processor 28.

In one embodiment, the processor 28 is operable to determine proximity of the transducer 17 to the imaging device 14. Radio frequency identification tag or other transponder may be used for identifying the proximity. For example, the signal strength or positional/ranging information of a wireless signal received by the receiver 32 is identified or measured. If the signal strength exceeds a threshold proximity, the transducer 17 and associated transducer probe 16 are likely adjacent to the imaging device 14, such as hung on a holder, and not in use. In response to exceeding threshold proximity, the processor 28 is operable to cause the imaging device 14 to cease driving the transducer 17 or cease generating transmit waveforms. By sensing the proximity of the transducer 17 to the imaging device 14, unnecessary heating of the transducer may be avoided. In this embodiment, the electronic identifier 20, another transponder or accelerometers are provided within the transducer probe 16. Variations in the signal characteristic may infer probe usage or lack of usage, such as where the probe is maintained in one position due to non-use. By sensing use, the power to the transducer 17 may be turned-on as well as turning off power when idle. Where multiple transducer probe assemblies 12 are connected with the imaging device 14 at a same time, the transducer probe 16 associated with signal variation or a non-close proximity is powered while the other transducer probes are unpowered, and the imaging device 14 is configured for use with the desired transducer probe 16 without further user selection.

In an alternative or additional embodiment, the processor 28 is operable to compare the code received from the electronic identifier 20 with security information. Unauthorized use of the transducer 17 or transducer assembly 12 is identified. The memory 30, such as a RAM, cache, hard drive, removable media or other memory, stores the security information, such as storing a code or list of codes associated with transducer assemblies 12.

By comparing the code received from the transducer assembly 12 with the security information, the processor is operable to generate a security signal. For example, in response to receiving an electronic identification from the electronic identifier 20, the processor 28 is operable to generate a security signal enabling use of the transducer 17 with the imaging system 14 or enabling security measures. For example, the processor 28 sets a flag in a database, generates an output signal, generates a signal altering the processing, generates a signal altering process flow or generates signals resulting in other changes within the processor 28, the transducer assembly 12, and/or the imaging device 14.

In one embodiment, a list of security information is provided for matching with an electronic identification. The processor 28 is operable to search for a match between the electronic identification within the list for determining authorized use. In one embodiment, the list is stored within the imaging device 14, such as a list of transducer probe assemblies 12 that may be used with the imaging device 14. The list of security information is input through the input 26. For example, the list stored on the imaging device 14 is input on the input 26. In another embodiment, the processor 28 or a different processor is provided within the transducer probe assembly 12 for identifying a list of imaging devices 14 usable with a given transducer probe assembly 12. The list of security information, such as the authorized imaging devices 14 to be stored on the transducer probe assembly 12 is input through the input 26 and programmed while connected to the imaging device. Alternatively, the transducer probe assembly 12 is programmed at the factory, programmed while connected to a computer, programmed wirelessly or removable media is provided to the transducer probe assembly 12 for programming.

When the transducer probe assembly 12 is connected to the imaging device, information, such as the code or other identifying information, is exchanged or queried. For example, the transducer probe assembly 12 queries or reads from the imaging device 14 for a serial number or vice versa. Through customer control of the security information, the transducer probe assembly 12 or the imaging device 14 determines whether use is authorized using a serial number, pass code or other identifying code. A password may then be used for initial entry or updating of the lists of security information as additional probes are purchased or moved between facilities.

In another embodiment, the security information is solicited for comparison with the code from the electronic identifier 20. For example, the processor 28 generates a request for security information or other code information in response to connection of the transducer probe assembly 12 with the imaging device 14. The user then inputs the code on the input 26. Each time the transducer probe assembly 12 connects to the imaging device 14, a request is generated. Alternatively, a match is used to program the transducer probe assembly 12 or the imaging device 14 to allow use without the request for further security information for future connections. For example, the processor 28 of the imaging device 14 identifies the code provided by the electronic identifier 20 as having been previously activated and allows a subsequent activation. Alternatively, the transducer probe assembly 12 receives identification information from the imaging device 14 and recognizes previously authorized use.

In response to a match of the entered code with the code from the transducer, the processor 28 is operable to allow use of the transducer assembly 12 with the imaging system 14. If a mismatch of the electronic identifier from the transducer probe assembly 12 in the entered code occurs, a security signal is generated. In one embodiment, the code from the electronic identifier 20 is the serial number or a different code and is directly matched with the input from the user. In other embodiments, the input from the user is altered in a preprogrammed manner, such as through encryption, for the comparison and matching. For example, a site code is entered and used to identify security information for the comparison without the site code having been programmed into the transducer probe assembly 12. In an alternative embodiment, a dipswitch or other input on the transducer assembly 12 is used for inputting the security information. A processor, such as the processor 28, within the transducer probe assembly 12 or the imaging device 14 performs the comparison and subsequent generation of security signal information.

Further programming within the transducer probe assembly 12 may allow for limited use of the transducer, such as limiting the number of uses of a catheter-based transducer as disclosed in U.S. Pat. No. 6,270,460, the disclosure of which is incorporated herein by reference. As an alternative to counting the number of uses, a duration of use may also be used to determine when a transducer be disabled until further sterilization and requalification. Using activation as discussed herein or reactivation by the manufacturer, a shutoff timer is enabled or reset for another usage. By providing the codes discussed herein, a manufacturer may assure safety requirements for sterilization and requalification are met. By charging for sterilization and requalification, the initial cost of the transducer may be reduced, resulting in the transducer cost being spread out over time. After each sterilization or requalification of the probe, a passcode is provided. If an unauthorized sterilization or requalification is performed, the passcode may not be known or provided. As a result, the transducer probe may not be subsequently used. The passcode rotates for each sterilization and requalification to avoid reentry of previous passcodes. The rotation of the passcode is programmed within the electronic identifier 20. The activation using a passcode then resets the count for the number of uses per sterilization and requalification. Other invasive products, such as transesophegeal probes, may be subject to similar control.

Tamper sensing or evidence markers or sensors may also be incorporated within the transducer probe assembly to assure that the product is not altered or otherwise tampered, such as caused by attempts to gain unauthorized access to the programming or identification information. Sensing attempts at alteration may be used to output a wrong code or no code, resulting in security activation.

In another embodiment, a comparison of code and security information is provided through an adapter 36 as shown in FIG. 2. The adapter 36 is connectable between the transducer probe assembly 12 and the imaging device 14. Alternatively, the adapter 36 is separately connectable with just the transducer probe assembly 12 or just the imaging device 14. While shown as a physical connection, the adapter 36 may be wirelessly connected to one or both of the transducer probe assembly 12 and the imaging device 14. The adapter 36 includes a memory and a connector 38 for connecting with the imaging device 14 in the transducer probe assembly 12. The memory is operable to store security information. A processor, such as the processor 28, is provided within the adapter 36 or at least partially within the adapter 36. The processor is operable to allow the use of the transducer 17 with the imaging device 14 in response to a match of security information, a list of codes, a single code or other information with an electronic identifier provided by the electronic identifier 20 and/or the imaging device 14. The adapter 36 acts to match the transducer probe 16 with the imaging device 14. Once the three sets of information are matched, the transducer probe assembly 12, the imaging system 14 or both are activated for current and later use together. For later uses, the transducer probe assembly 12 is connected with the imaging device 14 with or without the adapter 36. A flag, code or other signal is stored within the imaging device 14 or the transducer probe assembly 12 for registering connection between the same two devices to activate later use. Alternatively, each subsequent use requires the adapter 36. In one embodiment, the adapter 36 is associated with a facility, allowing programming of a list of different transducer probe assemblies 12 and imaging devices 14 associated with the facility. The adapter 36 is then used for initial connections of any new components to mediate activation.

Where a mismatch occurs, the security signal is generated to provide any of various theft prevention procedures. In one embodiment, the transducer probe assembly 12 is disabled. For example, an identification code provided by the electronic identifier is turned or switched off. In response to an incorrect identification code, the imaging device 14 prevents use of the ultrasound imaging transducer 12. Until a match confirms activation, the transducer probe assembly 12 may not output the identification of the type of transducer, resulting in the imaging device 14 being unable to use the transducer probe assembly 12. As another example, switches or other electronic processing is activated within the transducer probe assembly 12 to prevent signals from being transmitted to or being transmitted from the transducer array 17. The transducer probe assembly 12 is prevented from use with the particular imaging device 14 or any future uses until serviced. Software within the imaging device 14 may be used to disable use of the imaging device 14 with the transducer probe assembly 12. For example, the imaging device 14 is locked from use by the user, does not generate transmit waveforms, does not receive or process information or otherwise is made ineffective or less effective. The imaging device 14 is locked or disabled for use with the particular transducer probe assembly 12 or for all uses until serviced.

As an alternative or precursor to preventing use of the transducer probe assembly 12 or the imaging device 14, warning signals may be provided. For example, the processor 28 generates a display of a statement of ownership (e.g., “This probe is the property of Hospital”) or a warning (e.g., “Use of this transducer probe assembly 12 is unauthorized”). As another example, the ultrasound transducer probe assembly 12 is loaned to another facility for a limited number of uses. A warning is generated indicating the number of uses still available and the ownership of the probe. In one embodiment, a warning signal or other display statement is provided prior to disabling the system, such as allowing one or more emergency uses before a permanent disablement. The warnings may be generated without ever providing any disablement. The warning may be generated in conjunction with providing disablement (e.g., “This transducer probe is the property of Hospital and its use is unauthorized. The probe is stolen property and no longer usable”).

The level of security is set at the factory or within the software. Alternatively, the user is allowed to select the severity. The user programs the transducer probe assembly 12 to just issue a warning, to allow a certain number of unauthorized or authorized uses, to issue a warning with subsequent disablement, to disable or combinations thereof. The user may be allowed to program the language or wording of the displayed warning.

FIG. 3 shows a method for identifying unauthorized use of a transducer which is detachably connectable with an imaging device. The method uses the systems described above in FIGS. 1 or 2 or other now known or later developed systems. Additional, different or fewer acts than shown in FIG. 3 may be used in a same or different order.

In act 50, a transducer code is provided external to the transducer. For example, a transducer code stored on a transducer is wirelessly transmitted or transmitted through a wired connection to an imaging device. In alternative embodiments, a code from an imaging device is provided to the transducer. The code is a serial number in one embodiment, but may be a separate security code in other embodiments, such as a password or passcode.

In act 52, security information is input. In one embodiment, the security information is input by a user. Alternatively, the security information is input in a machine-readable form. The security information is input by a user in one embodiment in response to a request for security information. In another embodiment, an adapter is connected between the transducer and the imaging device for inputting the security information. In yet another embodiment, the security information was previously input and automatically provided without generation of a request for the security information. The security information is input to the transducer, the imaging device or an adapter.

In act 54, the transducer code is compared with the security information. For example, the transducer code is an identifier distinguishing the transducer from other transducers of a same type and manufacturer. The security information is a security code that matches the transducer code. Alternatively, the security information is a password that matches a transducer code other than an identifier. The same transducer code may be used for multiple transducers, such as programming all of the transducers at a facility or associated with a particular sonographer or a doctor with a password.

In one embodiment, the comparison is performed within the imaging device. In other embodiments, the comparison is performed within the transducer. In yet other embodiments, the comparison is performed in an external device, such as an adapter. For example, security information is provided from an imaging device to a transducer. The transducer than compares the transducer code with the security information. In another example, the transducer code is provided to the imaging device. The transducer code is compared to a list stored on the imaging device. The list of transducer identifiers allows matching of usable transducers with the imaging device. Alternatively, an imaging device code is matched with a list of usable imaging devices in the transducer. As yet another example, both the transducer code and imaging device code are matched with security information in an adapter.

In act 56, a security signal is generated. For example, a signal is generated indicating a match or authorized use. Alternatively, no signal is generated in response to authorized use. As another example, a mismatch of the transducer or imaging device code with the security information is identified. The security signal is then generated in response to the mismatch. The use of the transducer, the use of the imaging device, the use of the particular transducer with the particular imaging device or combinations thereof is disabled in response to a mismatch. Alternatively or additionally, a display of ownership, a warning or combinations thereof is generated in response to the mismatch. As a result, theft of transducer probe assemblies may be discouraged.

FIG. 4 shows one embodiment of a method for transducer identification. The transducer is electronically identified by an imaging device. The systems described above in FIGS. 1 or 2 or other now known or later developed systems may be used with the method of FIG. 4. Different, additional or fewer acts may be provided in the same or different order than shown in FIG. 4.

In act 60, identification information is wirelessly transmitted from a transducer. For example, the identification information is transponded in response to a field generated by an imaging device or other source. The identification information identifies a type of transducer, a specific transducer, or other data associated with the transducer or a group of transducers. For example, the identification information is a code distinguishing the transducer from other transducers of a same type and/or manufacturer.

In act 62, the identification information is received wirelessly. The information is received in an imaging device or a different device remote from the imaging device.

In act 64, the identification information is compared, such as comparing identification information identifying a type of transducer with a list of types of transducers for configuring the system. Additionally or alternatively, the identification information is compared with security information as discussed above.

In one embodiment, information wirelessly transmitted from the transducer is used to determine the proximity of the transducer to an imaging device. If a threshold proximity is exceeded, the transducer is determined to be spaced from or spaced adjacent to the imaging device. When the transducer is spaced adjacent to the imaging device and connected with the imaging device, the transducer is likely not being used. Transmit waveforms or driving of the transducer is ceased in response to the proximity or wireless information indicating a threshold proximity has been crossed. Where the proximity information indicates possible use by being spaced from the imaging device or spaced within a range of proximity from the imaging device associated with the position of patients, the imaging device is activated or allowed to continue generation of transmit waveforms driving the transducer. A radio frequency tag or other information used with the imaging device provides the wireless information indicating proximity. In one embodiment, the transmitted wireless information includes the proximity measurement or calculation performed by the transducer. In other embodiments, the received wireless information is measured or used to calculate the proximity by the imaging device.

As an alternative to determining proximity, the transducer assembly includes a positioning device, such as a local positioning receiver usable with a pseudolite. One or more pseudolites transmit coded ranging signals. The positioning device determines a location from the signals. The pseudolites act as unmoving satellites in global positioning system. The position information is then transmitted to the imaging device. A report or other list of transducers and the locations of the transducers is then generated. The positioning and identification information may be transmitted directly to or via the imaging device to a central location to provide a list of transducers and transducer locations within a facility or multiple facilities. A transducer with a homing device may be used so that the transducer could be located within the hospital. For example, the transmission from the transducer is triangulated using multiple receivers within the hospital.

The wireless device allows the transducer identification to be read remotely. The wireless communication may also allow the imaging system to determine the type or specific transducers near or available to the imaging system. When a transducer is connected with the imaging system, the type of transducer or the specific identification of the transducer wirelessly communicated to the imaging system is used to configure the imaging system for optimizing imaging performance. For example, the imaging frequency, aperture control, examination type or other operational software is optimized or selected. Specific transducer identification allows optimization or selection of parameters associated with the specific transducer, such as providing calibration relative to transducers of a same type that is distinct for each transducer. Imaging or other configuration software parameters may also be selected for use based on the specific transducer or the type of transducer wirelessly identified.

Other information may be transmitted, such as the number of uses, time of last use, time of all uses, user-specific identification information, the last settings used with the transducer, previously selected settings used with the transducer or other information. By providing user-specific, patient-specific or transducer-specific information, a workflow of a sonographer may be improved. The imaging system automatically fills in patient information, sonographer information or transducer-specific information for a particular exam to the wireless receiver for report generation. For example, if a transducer is later found to be defective, the patients for which a transducer was used are easily identified by the transducer identification stored with the patient record.

Asset management is provided by knowing what transducers are used, when they are used, when they are not used and how often they are used. Inventory management may also be provided using the wireless transmitter. During manufacturing, service or use by customers, the transducers are more easily monitored or tracked using wireless transmission. The wireless transmission acts as a barcode to improve inventory management through quick identification of a transducer, access to repair history information associated with the particular probe or identification of available probes for use. The removal of a probe from an immediate area may also be determined by loss of signal. The removal may result in generation of an alarm or other security signal.

Other recorded events may be stored or transmitted wirelessly, such as detections of abnormal conditions. For example, shocks are detected using an accelerometer and wirelessly transmitting the detected information. An excess temperature, faulty processing or other abnormal conditions may also be transmitted. In one embodiment, the abnormal condition is detected while the transducer is not connected with the ultrasound system and while powered by a battery (e.g., capacitor, lithium or other now known or later developed battery). In other embodiments, the abnormal condition is detected while the transducer is connected with the ultrasound system. A past or present warning is then generated based on the transmitted information. For example, a warning on the display prompts the sonographer to call for service due to possible damage to the transducer.

Other data may be wirelessly transmitted. For example, micro-code is transmitted for operating the imaging device. As yet another example, an amount of time or number of uses of the transducer is transmitted to a remote location. The frequency or utilization information may be used to identify appropriate training for optimized use of the most frequently used transducers, manage transducer use by multiple technicians, and recommend additional transducer purchases.

In one embodiment, a method for electronically identifying transducer information is provided. The transducer information is provided once the transducer is connected to the imaging device or once the transducer is within a wireless transmission range of the imaging device. An improvement is provided where the transducer is electronically distinguished from other transducers of a same type and manufacturer as discussed above. Specific information may be used for maintenance, transducer-specific calibration or configuration, security or other uses discussed herein, now known or later developed. An identifier unique to the transducer, such as an identifier with a low repeat rate relative to a volume of transducers of a same type or a same manufacturer, is used. The identifier is wirelessly transmitted or transmitted using a hardwire connection with or without one or more switches.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for identifying unauthorized use of a transducer which is detachably connectable with an imaging device, the system comprising:
an electronic identifier associated with the transducer; and
a processor operable to generate a security signal in response to the electronic identifier.

2. The system of claim 1 wherein the electronic identifier comprises an identifier distinguishing the transducer from other transducers of a same type and manufacturer.

3. The system of claim 1 wherein the electronic identifier comprises a memory storing a code.

4. The system of claim 1 wherein the processor comprises an encoder within the imaging device.

5. The system of claim 1 further comprising:
a memory operable to store a list of transducer identifiers including the electronic identifier; and
a connector operable to electronically connect the electronic identifier with the processor;
wherein the processor is operable to search for the electronic identifier within the memory.

6. The system of claim 5 further comprising:
an input on the imaging device, the input operable to receive the transducer identifiers.

7. The system of claim 1 wherein the security signal corresponds to one of a statement of ownership, a warning, prevention of use and combinations thereof.

8. The system of claim 1 further comprising:
a memory of the imaging device operable to store a code;
wherein the processor is operable to allow use of the transducer with the imaging system in response to a match of the code and the electronic identifier and is operable to generate the security signal in response to a mismatch of the codes and the electronic identifier.

9. The system of claim 8 further comprising:
an input on the imaging device, the input operable to receive the code;
wherein the processor is operable to generate a request for the code in response to connection of the transducer with the imaging device.

10. The system of claim 1 further comprising:
an adaptor connectable between the transducer and the imaging device, the adaptor having a memory operable to store security information;
wherein the processor is operable to allow use of the transducer with the imaging system in response to a match of the security information and the electronic identifier and is operable to generate the security signal in response to a mismatch of the security information and the electronic identifier.

11. The system of claim 1 wherein the electronic identifier comprises a radio frequency tag.

12. The system of claim 11 wherein the processor is operable to cause the imaging system to cease driving the transducer in response to proximity of the radio frequency tag.

13. A method for identifying unauthorized use of a transducer which is detachably connectable with an imaging device, the method comprising:
(a) comparing a code with a security code; and
(b) generating a security signal in response to the comparison.

14. The method of claim 13 wherein the code comprises a transducer identifier distinguishing the transducer from other transducers of a same type and manufacturer.

15. The method of claim 13 wherein the code is stored on the transducer;
further comprising:
(c) providing the code external to the transducer for (a).

16. The method of claim 13 further comprising:
(c) storing a list of transducer identifiers in the imaging device, the security code comprising the list;
wherein (a) comprises searching for the code within the list.

17. The method of claim 13 wherein (a) comprises identifying a mismatch of the code to the security information, and wherein (b) comprises generating a display of one of ownership, a warning and combinations thereof in response to the mismatch.

18. The method of claim 13 wherein (a) comprises identifying a mismatch of the code to the security information, and wherein (b) comprises disabling use of the transducer with the imaging device.

19. The method of claim 13 further comprising:
(c) generating a request for the security information; and
(d) inputting by a user the security information in response to the request;
wherein (b) comprises generating the security signal in response to a mismatch of the code and the security information.

20. The method of claim 13 further comprising:
(c) connecting an adaptor between the transducer and the imaging device;
wherein (a) comprises comparing the code from the transducer with the security information from the adaptor; and
wherein (b) comprises generating the security signal in response to a mismatch of the security information and the transducer code.

21. The method of claim 13 further comprising:
(c) wirelessly transmitting the code from the transducer to the imaging device;
wherein (a) comprises comparing in the imaging device.

22. The method of claim 21 further comprising:
(d) ceasing driving of the transducer in response to proximity of the radio frequency tag with the imaging device.

23. A system for transducer identification, the system comprising:
a transducer; and
a wireless identifier tag connected with the transducer.

24. The system of claim 23 wherein the transducer comprises a connector operable to releasably attach to an imaging device, and wherein the wireless identifier tag comprises a transponder having a code identifying a type of transducer.

25. The system of claim 23 wherein the wireless identifier tag comprises a transmitter and a memory storing a code distinguishing the transducer from other transducers of a same type and manufacturer.

26. The system of claim 25 further comprising:
an imaging device releasably connectable with the transducer, the imaging device having a receiver operable to receive the code from the transmitter, having an input operable to receive security information and having a processor operable to compare the code with the security information.

27. The system of claim 23 wherein the wireless identifier tag comprises a transmitter and a memory storing a code.

28. The system of claim 23 further comprising:
an imaging device having a processor operable to determine a threshold proximity of the transducer to the imaging device as a function of a signal from the wireless identifier tag, the imaging device operable to cease generation of transmit waveforms in response to the threshold proximity.

29. In a method for transducer identification wherein a transducer is electronically identified by an imaging device, the improvement comprising:
(a) transmitting identification information wirelessly from the transducer.

30. The improvement of claim 29 wherein the transducer comprises a connector operable to releasably attach to the imaging device, and wherein (a) comprises transponding the identification information in response to a field generated by the imaging device, the identification information identifying a type of transducer.

31. The improvement of claim 29 wherein (a) comprises transmitting a code distinguishing the transducer from other transducers of a same type and manufacturer.

32. The improvement of claim 29 further comprising:
(b) wirelessly receiving the identification information;
(c) inputting by a user security information; and
(d) comparing the identification information with the security information.

33. The improvement of claim 29 further comprising:
(b) determining a threshold proximity of the transducer to the imaging device as a function of wireless information; and
(c) ceasing generation of transmit waveforms in response to the threshold proximity.

34. In a method for electronically identifying transducer information, an improvement comprising:
(a) electronically distinguishing the transducer from other transducers of a same type and manufacturer.

35. The improvement of claim 34 wherein (a) comprises wirelessly transmitting an identifier unique to the transducer.

36. The improvement of claim 29 further comprising:
(b) receiving the identification information at a remote location other than the imaging device.

37. The improvement of claim 29 further comprising:
(b) transmitting one of frequency of use, position, micro-code and combinations thereof.

38. The improvement of claim 29 further comprising:
(b) performing asset management as a function of the identification information.

39. The improvement of claim 29 further comprising:
(b) transmitting transducer position information.

40. The improvement of claim 29 further comprising:
(b) generating a list of available transducers including the transducer as a function of wireless transmission of the identification information.

41. The improvement of claim 29 further comprising:
(b) selecting one of imaging parameters, calibration parameters, configuration parameters and combinations thereof as a function of the wirelessly transmitted identification information.

42. The improvement of claim 29 further comprising:
(b) detecting a variation in proximity as a function of a wireless signal strength; and
(c) activating use of the transducer by the imaging system in response to (b).

43. The improvement of claim 29 further comprising:
(b) detecting an abnormal condition of the transducer; and
(c) wirelessly transmitting an occurrence of the abnormal condition.

44. The improvement of claim 35 further comprising:
(b) storing the identifier in a patient record.

45. The improvement of claim 29 further comprising:
(b) maintaining one of asset and inventory management records as a function of the identification information.
